(19) United States
(12) Patent Application Publication
(10) Pub. No.: US 2026/0080535 A1
(43) Pub. Date: Mar. 19, 2026

(54) METHOD FOR CALCULATING DEMENTIA-RELATED INFORMATION USING VOLUME PREDICTED BY BRAIN CT AND ANALYSIS DEVICE THEREOF

(71) Applicant: SAMSUNG LIFE PUBLIC WELFARE FOUNDATION, Seoul (KR)

(72) Inventors: Chae Jung PARK, Seoul (KR); Soo Jong KIM, Seoul (KR); Yun A GU, Seoul (KR); Sang Won SEO, Seoul (KR); Hye Min JANG, Seoul (KR)

(21) Appl. No.: 19/398,339

(22) Filed: Nov. 24, 2025

(30) Foreign Application Priority Data
Jun. 27, 2023 (KR) 10-2023-0082852
Mar. 11, 2024 (WO) PCT/KR2024/003074

(51) Int. Cl. G06T 7/00; G06T 7/11; G16H 10/60; G16H 30/40; G16H 50/20

(52) U.S. Cl. CPC G06T 7/0012; G06T 7/11; G16H 10/60; G16H 30/40; G16H 50/20; G06T 2207/10081; G06T 2207/20081; G06T 2207/30016

(57) ABSTRACT

A method for deriving dementia-related information using volume predicted from brain CT includes: a step of an analysis apparatus receiving a brain CT (Computed Tomography) image of a subject; a step of the analysis apparatus inputting the brain CT image into a pre-trained segmentation model to extract regions of interest; a step of the analysis apparatus inputting pixel information of the regions of interest into a pre-trained first learning model to predict the volume of at least one region among the regions of interest; and a step of the analysis apparatus inputting the volume of the at least one region into a pre-trained second learning model to derive dementia-related information of the subject.

### CROSS REFERENCE TO RELATED APPLICATIONS

This application is a Continuation of PCT International Application No. PCT/KR2024/003074, filed on Mar. 11, 2024, which claims priority to Korean Patent Application No. 10-2023-0082852, filed on Jun. 27, 2023, which are all hereby incorporated by reference in their entirety.

### BACKGROUND

### Technical Field

The following description relates to a technique for predicting dementia-related information of a subject using brain CT images.

### Description of the Related Art

Dementia refers to a syndrome that causes cognitive function impairment such as memory, language, and judgment. Alzheimer's disease is the most common type of dementia. Brain imaging such as MRI (Magnetic Resonance Imaging) and PET is used for Alzheimer's diagnosis.

The description of the related art should not be assumed to be prior art merely because it is mentioned in or associated with this section. The description of the related art includes information that describes one or more aspects of the subject technology, and the description in this section does not limit the invention.

### SUMMARY

In one general aspect, there is provided a method of calculating dementia-related information using volume predicted from brain CT including: a step of an analysis apparatus receiving a brain CT image of a subject; a step of the analysis apparatus inputting the brain CT image into a pre-trained segmentation model to extract regions of interest; a step of the analysis apparatus inputting pixel information of the regions of interest into a pre-trained first learning model to predict the volume of at least one region among the regions of interest; and a step of the analysis apparatus inputting the volume of the at least one region into a pre-trained second learning model to derive dementia-related information of the subject.

In another general aspect, there is provided analysis apparatus for calculating dementia-related information using volume predicted from brain CT including: an interface device for receiving a brain CT image of a subject; a storage device storing a segmentation model for extracting regions of interest from brain CT images, a first learning model for receiving brain region of interest information and predicting volume, and a second learning model for calculating dementia-related information; and a computing device that inputs the received brain CT image into the segmentation model to extract regions of interest, inputs pixel information of the extracted regions of interest into the first learning model to predict the volume of at least one region among the regions of interest, and inputs the volume of the at least one region into the second learning model to derive dementia-related information of the subject.

Additional features, advantages, and aspects of the present disclosure are set forth in part in the description that follows and in part will become apparent from the present disclosure or may be trained by practice of the inventive concepts provided herein. Other features, advantages, and aspects of the present disclosure may be realized and attained by the descriptions provided in the present disclosure, or derivable therefrom, and the claims hereof as well as the drawings. It is intended that all such features, advantages, and aspects be included within this description, be within the scope of the present disclosure, and be protected by the following claims. Nothing in this section should be taken as a limitation on those claims. Further aspects and advantages are discussed below in conjunction with embodiments of the present disclosure.

It is to be understood that both the foregoing description and the following description of the present disclosure are examples, and are intended to provide further explanation of the disclosure as claimed.

### BRIEF DESCRIPTION OF THE DRAWINGS

The accompanying drawings, which are included to provide a further understanding of the present disclosure, are incorporated in and constitute a part of this present disclosure, illustrate aspects and embodiments of the present disclosure, and together with the description serve to explain principles and examples of the disclosure. In the drawings:

FIG. 1 is an example of a system for calculating dementia-related information based on CT images.

FIG. 2 is an example of a process for calculating dementia-related information using brain CT images.

FIG. 3 is an example of a process for training a segmentation model that extracts regions of interest from brain CT images.

FIG. 4 is an example of a training process for a learning model that predicts the volume of regions of interest based on extracted regions of interest.

FIG. 5 is an example of a training process for a learning model that derives dementia-related information based on brain region of interest volume.

FIG. 6 is an example of an analysis apparatus that derives dementia-related information of a subject using brain CT images.

Throughout the drawings and the detailed description, unless otherwise described, the same drawing reference numerals should be understood to refer to the same elements, features, and structures. The sizes of regions and elements, and depiction thereof may be exaggerated for clarity, illustration, and/or convenience.

### DETAILED DESCRIPTION

The following detailed description is provided to assist the reader in gaining a comprehensive understanding of the methods, apparatuses, and/or systems described herein. Accordingly, various changes, modifications, and equivalents of the systems, apparatuses and/or methods described herein will be understood by those of ordinary skill in the art.

Moreover, descriptions of well-known functions and constructions may be omitted for increased clarity and conciseness. Further, repetitive descriptions may be omitted for brevity. The progression of processing steps and/or operations described is a non-limiting example.

The sequence of steps and/or operations is not limited to that set forth herein and may be changed to occur in an order that is different from an order described herein, with the exception of steps and/or operations necessarily occurring in a particular order. In one or more examples, two operations in succession may be performed substantially concurrently, or the two operations may be performed in a reverse order or in a different order depending on a function or operation involved.

Unless stated otherwise, like reference numerals may refer to like elements throughout even when they are shown in different drawings. Unless stated otherwise, the same reference numerals may be used to refer to the same or substantially the same elements throughout the specification and the drawings. In one or more aspects, identical elements (or elements with identical names) in different drawings may have the same or substantially the same functions and properties unless stated otherwise. Names of the respective elements used in the following explanations are selected only for convenience and may be thus different from those used in actual products.

Advantages and features of the present disclosure, and implementation methods thereof, are clarified through the embodiments described with reference to the accompanying drawings. The present disclosure may, however, be embodied in different forms and should not be construed as limited to the embodiments set forth herein. Rather, these embodiments are examples and are provided so that this disclosure may be thorough and complete to assist those skilled in the art to understand the inventive concepts without limiting the protected scope of the present disclosure.

Shapes, dimensions (e.g., sizes, lengths, locations, and areas), proportions, ratios, numbers, the number of elements, and the like disclosed herein, including those illustrated in the drawings, are merely examples, and thus, the present disclosure is not limited to the illustrated details. It is, however, noted that the relative dimensions of the components illustrated in the drawings are part of the present disclosure.

When the term “comprise,” “have,” “include,” “contain,” “constitute,” “made of,” “formed of,” “composed of,” or the like is used with respect to one or more elements (e.g., components, structures, groups, circuits, networks, members, parts, areas, portions, integers, steps, operations, and/or the like), one or more other elements may be added unless a term such as “only” or the like is used. The terms used in the present disclosure are merely used in order to describe particular example embodiments, and are not intended to limit the scope of the present disclosure. The terms of a singular form may include plural forms unless the context clearly indicates otherwise. For example, an element may be one or more elements. An element may include a plurality of elements. The word “exemplary” is used to mean serving as an example or illustration. Embodiments are example embodiments. Aspects are example aspects. In one or more implementations, “embodiments,” “examples,” “aspects,” and the like should not be construed to be preferred or advantageous over other implementations. An embodiment, an example, an example embodiment, an aspect, or the like may refer to one or more embodiments, one or more examples, one or more example embodiments, one or more aspects, or the like, unless stated otherwise. Further, the term “may” encompasses all the meanings of the term “can.”

In one or more aspects, unless explicitly stated otherwise, an element, feature, or corresponding information (e.g., a level, range, dimension, or the like) is construed to include an error or tolerance range even where no explicit description of such an error or tolerance range is provided. An error or tolerance range may be caused by various factors (e.g., process factors, internal or external impact, noise, or the like). In interpreting a numerical value, the value is interpreted as including an error range unless explicitly stated otherwise.

When a positional relationship between two elements (e.g., components, structures, groups, circuits, networks, members, parts, areas, portions, and/or the like) are described using any of the terms such as “adjacent to,” “beside,” “next to,” and/or the like indicating a position or location, one or more other elements may be located between the two elements unless a more limiting term, such as “immediate(ly),” “direct(ly),” or “close(ly),” is used. Furthermore, the spatially relative terms such as the foregoing terms as well as other terms such as “column,” “row,” “vertical,” “horizontal,” “diagonal,” and the like refer to an arbitrary frame of reference.

In describing a temporal relationship, when the temporal order is described as, for example, “after,” “following,” “subsequent,” “next,” “before,” “preceding,” “prior to,” or the like, a case that is not consecutive or not sequential may be included and thus one or more other events may occur therebetween, unless a more limiting term, such as “just,” “immediate(ly),” or “direct(ly),” is used.

It is understood that, although the terms “first,” “second,” and the like may be used herein to describe various elements (e.g., components, structures, groups, circuits, networks, members, parts, areas, portions, and/or the like), these elements should not be limited by these terms, for example, to any particular order, precedence, or number of elements. These terms are used only to distinguish one element from another. For example, a first element may denote a second element, and, similarly, a second element may denote a first element, without departing from the scope of the present disclosure. Furthermore, the first element, the second element, and the like may be arbitrarily named according to the convenience of those skilled in the art without departing from the scope of the present disclosure. For clarity, the functions or structures of these elements (e.g., the first element, the second element, and the like) are not limited by ordinal numbers or the names in front of the elements. Further, a first element may include one or more first elements. Similarly, a second element or the like may include one or more second elements or the like.

In describing elements of the present disclosure, the terms “first,” “second,” “A,” “B,” “(a),” “(b),” or the like may be used. These terms are intended to identify the corresponding element(s) from the other element(s), and these are not used to define the essence, basis, order, or number of the elements.

The expression that an element (e.g., component, structure, group, circuit, network, member, part, area, portion, and/or the like) “is engaged” with another element may be understood, for example, as that the element may be either directly or indirectly engaged with the another element. The term “is engaged” or similar expressions may refer to a term such as “is connected,” “is coupled,” “is combined,” “is linked,” “is provided,” “interacts,” or the like. The engagement may involve one or more intervening elements disposed or interposed between the element and the another element, unless otherwise specified.

The terms such as a “line” or “direction” should not be interpreted only based on a geometrical relationship in which the respective lines or directions are parallel, perpendicular, diagonal, or slanted with respect to each other, and may be meant as lines or directions having wider directivities within the range within which the components of the present disclosure may operate functionally.

The term “at least one” should be understood as including any and all combinations of one or more of the associated listed items. For example, each of the phrases “at least one of a first item, a second item, or a third item” and “at least one of a first item, a second item, and a third item” may represent (i) a combination of items provided by two or more of the first item, the second item, and the third item or (ii) only one of the first item, the second item, or the third item. Further, at least one of a plurality of elements can represent (i) one element of the plurality of elements, (ii) some elements of the plurality of elements, or (iii) all elements of the plurality of elements. Further, “at least some,” “at least some portions,” “at least some parts,” “at least a portion,” “at least one or more portions,” “at least a part,” “at least one or more parts,” “at least some elements,” “one or more,” or the like of a plurality of elements can represent (i) one element of the plurality of elements, (ii) a portion (or a part) of the plurality of elements, (iii) one or more portions (or parts) of the plurality of elements, (iv) multiple elements of the plurality of elements, or (v) all of the plurality of elements. Moreover, “at least some,” “at least some portions,” “at least some parts,” “at least a portion,” “at least one or more portions,” “at least a part,” “at least one or more parts,” or the like of an element can represent (i) a portion (or a part) of the element, (ii) one or more portions (or parts) of the element, or (iii) the element, or all portions of the element.

The expression of a first element, a second elements “and/or” a third element should be understood as one of the first, second and third elements or as any or all combinations of the first, second and third elements. By way of example, A, B and/or C may refer to only A; only B; only C; any of A, B, and C (e.g., A, B, or C); some combination of A, B, and C (e.g., A and B; A and C; or B and C); or all of A, B, and C. Furthermore, an expression “A/B” may be understood as A and/or B. For example, an expression “A/B” may refer to only A; only B; A or B; or A and B.

In one or more aspects, the terms “between” and “among” may be used interchangeably simply for convenience unless stated otherwise. For example, an expression “between a plurality of elements” may be understood as among a plurality of elements. In another example, an expression “among a plurality of elements” may be understood as between a plurality of elements. In one or more examples, the number of elements may be two. In one or more examples, the number of elements may be more than two. Furthermore, when an element is referred to as being “between” at least two elements, the element may be the only element between the at least two elements, or one or more intervening elements may also be present.

In one or more aspects, the phrases “each other” and “one another” may be used interchangeably simply for convenience unless stated otherwise. For example, an expression “different from each other” may be understood as being different from one another. In another example, an expression “different from one another” may be understood as being different from each other. In one or more examples, the number of elements involved in the foregoing expression may be two. In one or more examples, the number of elements involved in the foregoing expression may be more than two.

In one or more aspects, the phrases “one or more among” and “one or more of” may be used interchangeably simply for convenience unless stated otherwise.

The term “or” means “inclusive or” rather than “exclusive or.” That is, unless otherwise stated or clear from the context, the expression that “x uses a or b” means any one of natural inclusive permutations. For example, “a or b” may mean “a,” “b,” or “a and b.” For example, “a, b or c” may mean “a,” “b,” “c,” “a and b,” “b and c,” “a and c,” or “a, b and c.”

A phrase “substantially the same” may indicate a degree of being considered as being equivalent to each other taking into account minute differences due to errors in the manufacturing or operating process.

Features of various embodiments of the present disclosure may be partially or entirely coupled to or combined with each other, may be technically associated with each other, and may be variously operated, linked or driven together in various ways. Embodiments of the present disclosure may be implemented or carried out independently of each other or may be implemented or carried out together in a co-dependent or related relationship. In one or more aspects, the components of each apparatus and device according to various embodiments of the present disclosure are operatively coupled and configured.

The terms used herein have been selected as being general in the related technical field; however, there may be other terms depending on the development and/or change of technology, convention, preference of technicians, and so on. Therefore, the terms used herein should not be understood as limiting technical ideas, but should be understood as examples of the terms for describing example embodiments.

Further, in a specific case, a term may be arbitrarily selected by an applicant, and in this case, the detailed meaning thereof is described herein. Therefore, the terms used herein should be understood based on not only the name of the terms, but also the meaning of the terms and the content hereof.

In the following description, various example embodiments of the present disclosure are described in more detail with reference to the accompanying drawings. With respect to reference numerals to elements of each of the drawings, the same elements may be illustrated in other drawings, and like reference numerals may refer to like elements unless stated otherwise. The same or similar elements may be denoted by the same reference numerals even though they are depicted in different drawings. In addition, for the convenience of description, a scale and dimension of each of the elements illustrated in the accompanying drawings may be different from an actual scale and dimension, and thus, embodiments of the present disclosure are not limited to a scale and dimension illustrated in the drawings.

Before starting detailed explanations of figures, components that will be described in the specification are distinguished merely according to functions mainly performed by the components. That is, two or more components which will be described later can be integrated into a single component. Furthermore, a single component which will be explained later can be separated into two or more components. Moreover, each component which will be described can additionally perform some or all of a function executed by another component in addition to the main function thereof. Some or all of the main function of each component which will be explained can be carried out by another component. Accordingly, presence/absence of each component which will be described throughout the specification should be functionally interpreted.

The technology described below is a technology for calculating dementia-related information from brain CT.

Hereinafter, dementia includes Alzheimer's dementia.

Dementia-related information refers to information related to dementia diagnosis or dementia prediction. For example, dementia-related information may be at least one of information such as dementia onset status, dementia risk, dementia probability, dementia-related score, dementia prognosis prediction, degree of brain atrophy, beta-amyloid (amyloid-β, Aβ) positivity, tau protein positivity, and brain age.

Hereinafter, a device that analyzes brain CT to derive dementia-related information for a subject is referred to as an analysis apparatus. The analysis apparatus may take the form of a computer device such as a PC, a smart device, a network server, or a data processing dedicated chipset.

The analysis apparatus may derive dementia-related information based on brain images using multiple learning models. The analysis apparatus may extract region(s) of interest from CT using a segmentation model. In addition, the analysis apparatus may derive dementia-related information from CT or region(s) of interest using a classification model.

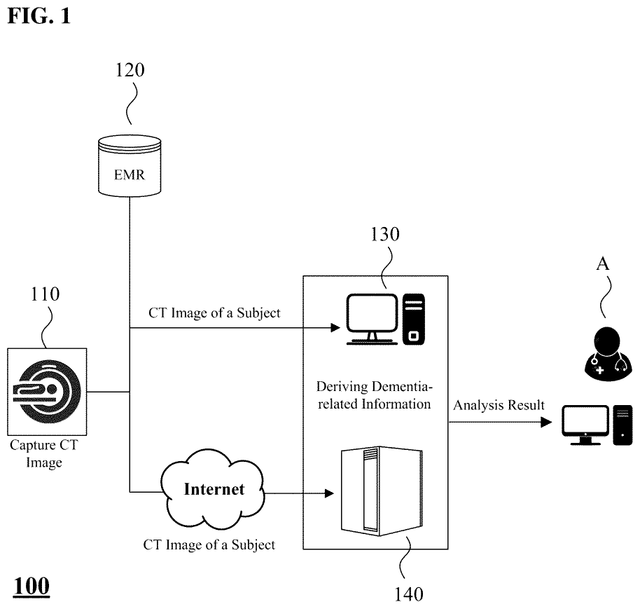

FIG. 1 is an example of a system (100) for calculating dementia-related information based on CT images. In FIG. 1, the analysis apparatus is shown as a computer terminal (130) and a server (140).

The CT imaging device (110) captures a CT image of a subject. The CT image is an image of the brain region (brain CT image). The brain CT image generated by the CT imaging device (110) may be stored in a separate database such as EMR (Electronic Medical Record, 120).

The computer terminal (130) can receive the subject's brain CT image from the CT imaging device (110) or EMR (120) through a wired or wireless network. In some cases, the computer terminal (130) may be a device physically connected to the CT imaging device (110). The computer terminal (130) may uniformly preprocess the brain CT image.

The computer terminal (130) extracts region(s) of interest from the brain CT image. The computer terminal (130) may extract region(s) of interest from the brain CT image using a pre-trained segmentation model. The computer terminal (130) may estimate the volume of the region(s) of interest using a learning model. The computer terminal (130) may derive dementia-related information for the subject based on the volume of the region(s) of interest using a learning model. User A may check dementia-related information through the computer terminal (130).

The server (140) may receive the subject's brain CT image from the CT imaging device (110) or EMR (120) through a wireless network. The server (140) may uniformly preprocess the brain CT image.

The server (140) extracts region(s) of interest from the brain CT image. The server (140) may extract region(s) of interest from the brain CT image using a pre-trained segmentation model. The server (140) may estimate the volume of the region(s) of interest using a learning model. The server (140) may derive dementia-related information for the subject based on the volume of the region(s) of interest using a learning model. The server (140) may transmit the dementia-related information to a user terminal. User A may check dementia-related information through the user terminal.

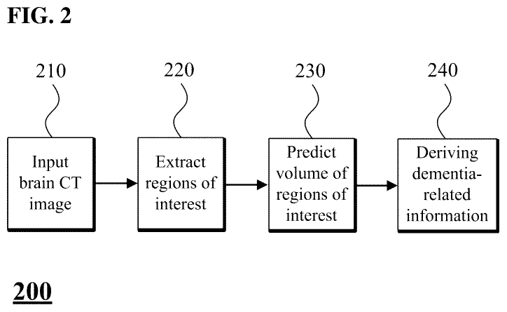

FIG. 2 is an example of a process (200) for calculating dementia-related information using brain CT images. The analysis apparatus may derive dementia-related information for a subject based on brain CT images.

The analysis apparatus receives a brain CT image of the subject (210). Brain CT may consist of multiple slices. The object analyzed by the analysis apparatus may be all of the multiple slices or at least one slice among the multiple slices.

The analysis apparatus may uniformly preprocess the brain CT image.

For example, the analysis apparatus may extract the entire brain region from the brain CT image. In this process, the analysis apparatus may use a pre-trained segmentation model. The analysis apparatus may extract the entire brain region from brain MRI images using the CIVET pipeline to generate ground truth, and train the segmentation model to extract the entire brain region from brain CT images based on the correct answer (entire brain region of brain MRI images). Thereafter, the analysis apparatus may extract the entire brain region from brain CT images using the trained segmentation model.

In addition, the analysis apparatus may normalize the size or resolution of the subject's brain CT image to a certain size.

The analysis apparatus extracts regions of interest from the received brain CT image (220). The analysis apparatus may extract regions of interest using a pre-trained segmentation model. The analysis apparatus may extract multiple regions of interest using a segmentation model. The regions of interest correspond to regions for calculating the volume of the corresponding region.

The regions of interest may include at least one of a cerebrospinal fluid (CSF) region and a ventricle region. The regions of interest may include at least one of frontal CSF, temporal CSF, parietal CSF, occipital CSF, anterior lateral ventricle, posterior lateral ventricle, and peri-hippocampal CSF.

The analysis apparatus may estimate the volume of the extracted region(s) of interest (230). The analysis apparatus may estimate the volume of a specific region of interest using a learning model (e.g., regression analysis model) that has pre-trained the relationship between the number of pixels for a specific region of interest and the volume of the corresponding region. For this purpose, the analysis apparatus needs to normalize the size of the brain CT image or region of interest uniformly. The analysis apparatus may estimate the volume of a specific region of interest based on a uniformly normalized brain CT image. At this time, the learning model may be prepared in advance for each of multiple regions of interest. For example, the analysis apparatus may individually estimate the volume of the corresponding region using learning models for each of the frontal cerebrospinal fluid region, temporal cerebrospinal fluid region, parietal cerebrospinal fluid region, occipital cerebrospinal fluid region, anterior lateral ventricle region, posterior lateral ventricle region, and peri-hippocampal cerebrospinal fluid region.

Meanwhile, the analysis apparatus may additionally use the subject's clinical information (gender, age, etc.) in addition to the brain CT image to estimate the volume of the region of interest.

The analysis apparatus may derive dementia-related information for the subject based on the volume of the region(s) of interest (240). The analysis apparatus may derive dementia-related information based on the volume of the region(s) of interest using a learning model. In addition, the analysis apparatus may derive the subject's dementia-related information by further using additional clinical information (age, gender, APOE4 genotype, etc.) in addition to the volume of the region(s) of interest.

In certain embodiments, the learning models are trained on datasets from a population who visited an affiliated medical institution (Samsung Seoul Hospital). The population consisted of dementia patients (Alzheimer's disease dementia, ADD) and normal controls (NC). The population was selected as subjects who had both MRI and CT imaging within a certain period (1 year). The population excluded normal people who were too young and also excluded dementia patients with cardiovascular diseases. The population data includes MRI, CT, clinical information (gender, age, APOE4 phenotype), and dementia status (label value). The total number of subjects in the population was 895. Table 1 below shows information about the population. In certain embodiments, datasets from 716 people (80% of 895 people) are used as training data and data from 179 people (20%) as test data. Validation was conducted with 5-fold cross validation.

TABLE 1

| | Total | | NC | | ADD | |
|---|---|---|---|---|---|---|
| Subjects, N (%) | 895 | (100.0) | 457 | (51.1) | 438 | (48.9) |
| Age, mean years | 67.4 | (11.4) | 66.8 | (12.5) | 68.0 | (10.2) |
| (SD, Standard Deviation) | | | | | | |
| Female, N (%) | 541 | (60.4) | 279 | (60.9) | 262 | (59.8) |
| APOEε4 carriers, N(%) | 322 | (36.0) | 86 | (18.8) | 236 | (51.5) |

| | | | |
|---|---|---|---|
| Frontal cerebrospinal fluid | 61052.5 | 50380.9 | 72187.0 |
| region, mean volume (mm³) | | | |
| Temporal cerebrospinal fluid | 36534.9 | 31169.5 | 42133.0 |
| region, mean volume (mm³) | | | |
| Parietal cerebrospinal fluid | 36045.5 | 29592.5 | 42778.3 |
| region, mean volume (mm³) | | | |
| Occipital cerebrospinal fluid | 14828.4 | 13150.0 | 16579.6 |
| region, mean volume (mm³) | | | |
| Anterior lateral ventricle | 17687.3 | 13955.5 | 21581.1 |
| region, mean volume (mm³) | | | |
| Posterior lateral ventricle | 20476.3 | 14904.4 | 26289.8 |
| region, mean volume (mm³) | | | |
| Peri-hippocampal cerebrospinal | 7577.0 | 5562.8 | 9678.6 |
| fluid region, mean volume (mm³) | | | |

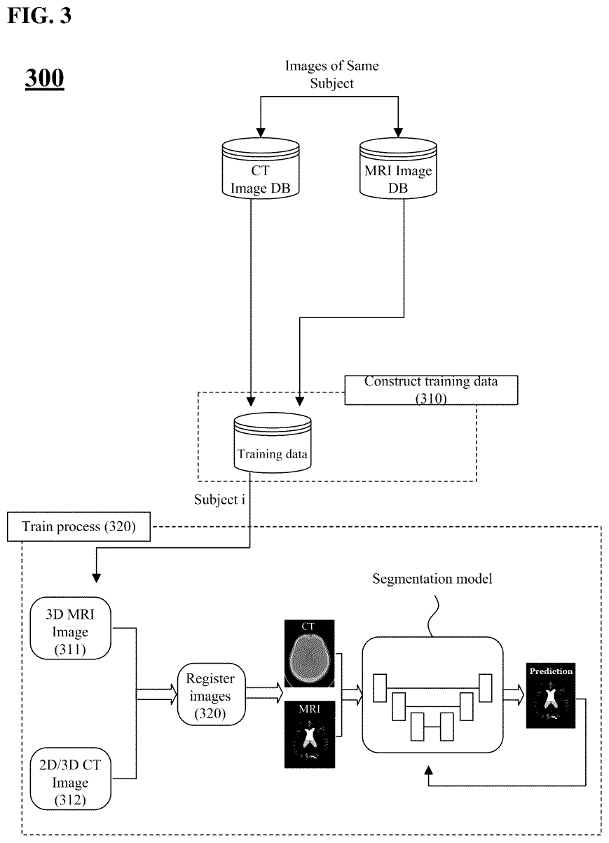

FIG. 3 is an example of a process (300) for learning a segmentation model that extracts regions of interest from brain CT images. In certain embodiments, normalized brain CT images are used as training data to a uniform size. In addition, augmented training data through brightness adjustment, horizontal flip, etc. are used as the training data.

Hereinafter, the training process of the model is described as being performed by a) learning apparatus. In certain embodiments, a learning apparatus trains the segmentation model. The learning apparatus refers to a computer device capable of processing image data and performing the training process of machine learning models.

The learning apparatus learns the segmentation model using medical images of multiple subjects. However, FIG. 3 describes as an example a process of learning a segmentation model using data from subject i.

The learning apparatus constructs training data for learning the learning model (310). The training data may be constructed from brain images of a population (including dementia patients and normal people). The CT image DB (database) stores brain CT images of the population. The MRI image DB stores brain MRI images of the population. The MRI image DB includes region of interest information in brain images. The region of interest information may be a region of interest automatically or manually labeled by an expert. The learning apparatus constructs training data by extracting CT images and MRI images (including region of interest information) for the same subject.

The learning apparatus performs a process of learning a segmentation model that extracts regions of interest using the training data (320). The learning apparatus performs learning based on data for a specific patient among the training data. The learning apparatus may repeatedly perform the training process using data for multiple patients in the training data.

The learning apparatus receives subject i's 3D MRI image (including region of interest information) (311). In addition, the learning apparatus receives subject i's 2D or 3D CT image (312). The region of interest may be an ROI in a 2D image or a VOI (Volume of Interest) in a 3D image. The learning apparatus registers the brain MRI image and brain CT image (320). The learning apparatus registers images of the same brain region of the same subject.

The learning apparatus constructs a segmentation model using the registered brain CT and brain MRI. The segmentation model may be a semantic segmentation model. For example, the segmentation model may be a FCN (Fully convolutional network)-based model such as U-net.

The segmentation model predicts regions of interest based on brain CT images. In the training process, the segmentation model is trained to extract the same regions of interest in brain CT images by referring to the regions of interest in brain MRI images that are ground truth.

The regions of interest may be multiple regions as described above. The regions of interest may include frontal cerebrospinal fluid region, temporal cerebrospinal fluid region, parietal cerebrospinal fluid region, occipital cerebrospinal fluid region, anterior lateral ventricle region, posterior lateral ventricle region, and peri-hippocampal cerebrospinal fluid region. The learning apparatus may construct a segmentation model for each region of interest.

In certain embodiments, multiple segmentation models for each region of interest are used. U-net-based segmentation models can be used. Researchers constructed a segmentation model (2D image-based segmentation model) using each of the axial slices in the training data. In addition, researchers constructed a segmentation model (3D image-based segmentation model) using 3D CT of the training data. Table 2 below shows the results of evaluating the performance of the constructed segmentation model. Researchers performed 5-fold cross-validation on the validation data to evaluate the performance of the 2D image-based segmentation model and 3D image-based segmentation model.

TABLE 2

| | | | | | | |
|---|---|---|---|---|---|---|
| | | | | Anterior | Posterior | |
| | | | | lateral | lateral | Peri- |
| Frontal | Temporal | Parietal | Occipital | ventricle | ventricle | hippocampal |
| CSF region | CSF region | CSF region | CSF region | region | region | CSF region |

2D image-based segmentation model 2D (n = 179)

| | | | | | | | |
|---|---|---|---|---|---|---|---|
| 1 Fold | 0.5686 | 0.4832 | 0.5711 | 0.4294 | 0.8848 | 0.8573 | 0.6511 |
| 2 Fold | 0.5682 | 0.4786 | 0.5686 | 0.4246 | 0.8833 | 0.8563 | 0.6476 |
| 3 Fold | 0.5815 | 0.4949 | 0.5813 | 0.4443 | 0.8816 | 0.8557 | 0.6442 |
| 4 Fold | 0.5836 | 0.4938 | 0.5835 | 0.4434 | 0.8822 | 0.8548 | 0.6435 |
| 5 Fold | 0.5711 | 0.4872 | 0.5714 | 0.4311 | 0.8849 | 0.8584 | 0.6613 |

3D image-based segmentation model (n = 179)

| | | | | | | | |
|---|---|---|---|---|---|---|---|
| 1 Fold | 0.5827 | 0.4885 | 0.5903 | 0.4449 | 0.8851 | 0.8603 | 0.6278 |
| 2 Fold | 0.5865 | 0.4907 | 0.5922 | 0.448 | 0.8837 | 0.859 | 0.6273 |
| 3 Fold | 0.594 | 0.4967 | 0.6037 | 0.4608 | 0.8832 | 0.8593 | 0.6201 |
| 4 Fold | 0.6026 | 0.5023 | 0.6063 | 0.4613 | 0.8823 | 0.8563 | 0.6181 |
| 5 Fold | 0.5864 | 0.4907 | 0.594 | 0.4494 | 0.8862 | 0.8611 | 0.6357 |

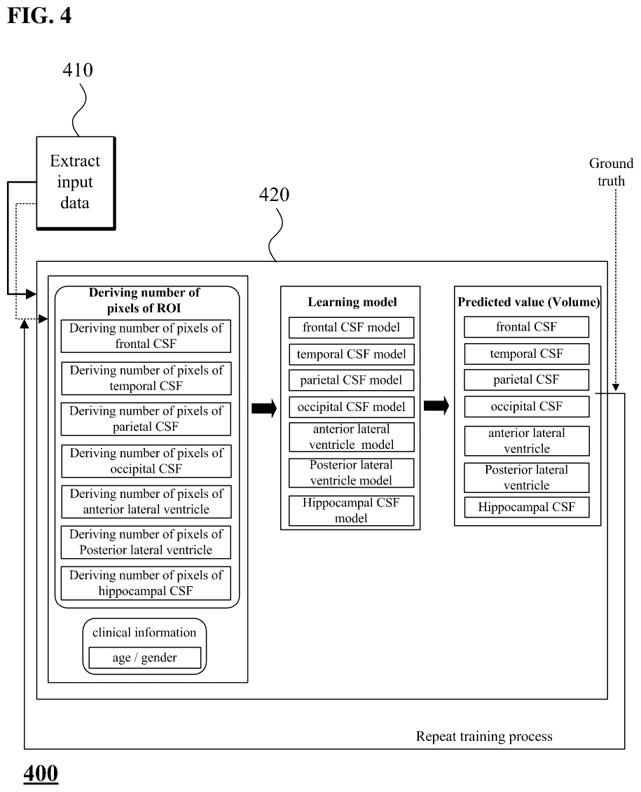

FIG. 4 is an example of a training process (400) of a learning model that predicts the volume of a region of interest based on the extracted region of interest. In FIG. 3, the learning model predicts the volume of the region of interest based on the aforementioned region of interest.

The learning in FIG. 4 presupposes a state in which the aforementioned regions of interest have been extracted from brain CT images of subjects belonging to the population. That is, the learning apparatus may use regions of interest derived using the segmentation model of FIG. 3.

The learning apparatus extracts input data based on regions of interest extracted from brain CT images (410). The input data includes region(s) of interest extracted by the segmentation model. The input data includes the actual volume (correct answer) of a specific region of interest. The actual volume may be derived from MRI. Furthermore, the input data may include clinical information (gender, age) about the subject.

The learning apparatus performs a process of learning a learning model using the extracted input data (420). The learning apparatus repeats the training process of the learning model using input data extracted from brain CT images of multiple subjects. The learning apparatus may construct a learning model that predicts volume for each region of interest.

The learning apparatus derives the total number of pixels for the region of interest. The regions of interest may be frontal cerebrospinal fluid region, temporal cerebrospinal fluid region, parietal cerebrospinal fluid region, occipital cerebrospinal fluid region, anterior lateral ventricle region, posterior lateral ventricle region, and peri-hippocampal cerebrospinal fluid region, respectively, as described above.

The learning apparatus constructs a learning model by comparing the number of pixels in the region of interest with the volume (correct answer) of the corresponding region of interest. Through this process, the learning model is trained to predict the volume of the region of interest. The correct answer is the volume of a specific region of interest derived from brain MRI of subjects belonging to the population.

The learning model may be constructed as individual models according to the information to be predicted. The learning model is a machine learning model and may be implemented as one of various types of models. Researchers used a regression model. Researchers individually constructed a model for predicting the volume of frontal CSF (frontal CSF model), a model for predicting the volume of temporal CSF (temporal CSF model), a model for predicting the volume of parietal CSF (parietal CSF model), a model for predicting the volume of occipital CSF (occipital CSF model), a model for predicting the volume of anterior lateral ventricle (lateral ventricle model), a model for predicting the volume of posterior lateral ventricle (posterior lateral ventricle model), and a model for predicting the volume of peri-hippocampal cerebrospinal fluid region (hippocampal CSF model).

Furthermore, the learning model may be trained using clinical information (age and gender) in addition to the size of the corresponding region of interest.

Researchers evaluated the performance of the constructed volume prediction model.

Researchers compared the predicted volume of regions of interest extracted from brain CT images with the aforementioned segmentation model with the correct answer measured from 3D MRI. Researchers segmented regions of interest using each of the two constructed segmentation models, and compared the results of predicting volume based on these results with the ground truth. The performance evaluation results of the volume prediction model are shown in Table 3 below. The performance indicator is the Pearson Correlation Coefficient. Researchers repeated cross validation 10 times with 10 permutations. The volume of the region of interest predicted by the learning model showed significant correlation with the actual correct answer.

TABLE 3

| | Correlation r | Correlation r |
|---|---|---|
| | (Volume prediction | (Volume prediction |
| | of region of interest | of region of interest |
| | segmented with 2D | segmented with 3D |
| Region of Interest | image-based) | image-based) |
| Frontal cerebrospinal | 0.838 | 0.868 |
| fluid region | | |
| Temporal cerebrospinal | 0.827 | 0.878 |
| fluid region | | |
| Parietal cerebrospinal | 0.868 | 0.905 |
| fluid region | | |
| Occipital cerebrospinal | 0.761 | 0.824 |
| fluid region | | |
| Anterior lateral | 0.984 | 0.984 |
| ventricle region | | |
| Posterior lateral | 0.981 | 0.981 |
| ventricle region | | |
| Peri-hippocampal | 0.945 | 0.944 |
| cerebrospinal | | |
| fluid region | | |

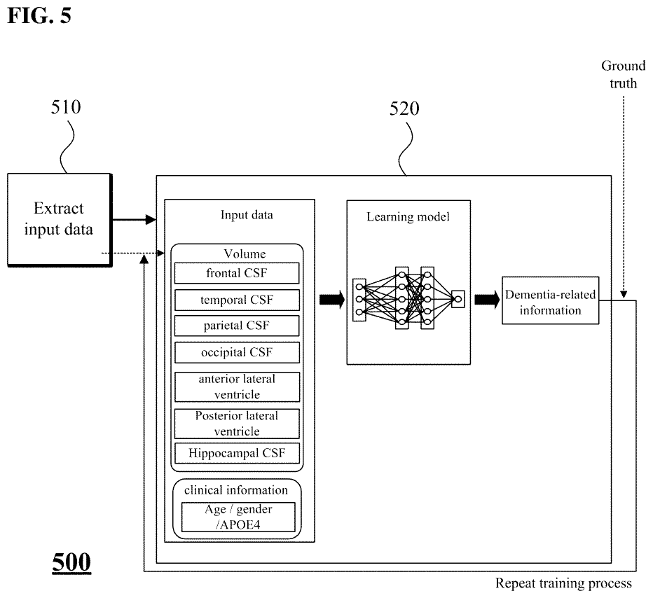

FIG. 5 is an example of a training process (500) of a learning model that derives dementia-related information based on brain region of interest volume. The learning model that derives dementia-related information can be implemented as one of various machine learning model types. Researchers implemented a model that derives dementia-related information as a deep learning model.

The learning in FIG. 5 presupposes a state in which the volume of brain regions of interest and clinical information for subjects belonging to the population have been obtained. For example, the learning apparatus may use the volume by region of interest of the subject derived using the learning model of FIG. 4.

The learning apparatus acquires input data that is training data (510).

The learning apparatus extracts the volume of a specific region of interest extracted from brain CT images of subjects belonging to the population. The brain region volume may include the volumes of frontal cerebrospinal fluid region, temporal cerebrospinal fluid region, parietal cerebrospinal fluid region, occipital cerebrospinal fluid region, anterior lateral ventricle region, posterior lateral ventricle region, and peri-hippocampal cerebrospinal fluid region. Furthermore, the learning apparatus may construct a learning model to derive dementia-related information based on some regions of interest among multiple regions of interest.

In addition, the learning apparatus acquires clinical information of subjects. Clinical information may include age, gender, and APOE4 phenotype. The learning apparatus may construct a learning model to derive dementia-related information by further using some information among clinical information in addition to the volume of region(s) of interest.

The training data may include the volume of a specific brain region derived using the learning model of FIG. 3. Alternatively, the training data may include cerebral cortex thickness and volume of specific brain regions derived from actual subjects' brain images. Researchers used the volume of regions of interest derived from 3D MRI analysis results of actual subjects as training data.

The learning apparatus performs a process of learning a learning model using the extracted input data (520). The learning apparatus repeats the training process of the learning model using input data extracted from multiple subjects.

The learning apparatus performs learning by inputting the extracted input data into the learning model and comparing the value output (predicted value) by the learning model with the correct answer. The learning model is trained to predict dementia-related information of the subject. At this time, the correct answer is the dementia-related information of the corresponding subject.

Researchers constructed a learning model that classifies dementia patients or normal people. Researchers used 90% of the population data as training data and 10% of the data as validation data. The multiple learning models constructed by researchers are shown in Table 4 below. The learning models were largely constructed as models using only the volume of regions of interest (Model A), models using the volume of regions of interest and clinical information (age and gender) (Model B), and models using the volume of regions of interest and clinical information (age, gender, and APOE4 phenotype) (Model C). Each model group was constructed by dividing into models using 1, 2, 3, 4, 6, and 7 (all) regions of interest among all regions of interest.

Table 4 shows model groups using 4 different ROIs and the training data used for constructing different models in each group. In Table 4 below, the model is a model that predicts whether the subject has dementia (dementia or normal). That is, it corresponds to a model in which the learning model in FIG. 4 performs binary classification of whether the subject has dementia or is normal.

TABLE 4

| Model | Model | |
|---|---|---|
| Classification | Name | Input Features |
| Model A | Model A1 | 3 ROIs (aLV/pLV/HippCSF) |
| | Model A2 | 4 ROIs (F/T/P/O) |
| | Model A3 | 6 ROIs (F/T/P/O/aLV/pLV) |
| | Model A4 | 7 ROIs (F/T/P/O/aLV/pLV/HippCSF) |
| | Model A5 | 2 ROIs (aLV/pLV) |
| | Model A6 | 1 ROIs (HippCSF) |
| Model B | Model B1 | 3 ROIs (aLV/pLV/HippCSF) + (age, gender) |
| | Model B2 | 4 ROIs (F/T/P/O) + (age, gender) |
| | Model B3 | 6 ROIs (F/T/P/O/aLV/pLV) + (age, gender) |
| | Model B4 | 7ROIs (F/T/P/O/aLV/pLV/HippCSF) + (age, gender) |
| | Model B5 | 2 ROIs (aLV/pLV) + (age, gender, APOE4) |
| | Model B6 | 1 ROIs (HippCSF) + (age, gender, APOE4) |
| Model C | Model C1 | 3 ROIs (aLV/pLV/HippCSF) + (age, gender, APOE4) |
| | Model C2 | 4 ROIs (F/T/P/O) + (age, gender, APOE4) |
| | Model C3 | 6 ROIs (F/T/P/O/aLV/pLV) + (age, gender, APOE4) |
| | Model C4 | 7 ROIs (F/T/P/O/aLV/pLV/HippCSF) + (age, gender, APOE4) |
| | Model C5 | 2 ROIs (aLV/pLV) + (age, gender, APOE4) |
| | Model C6 | 1 ROIs (HippCSF) + (age, gender, APOE4) |

In Table 4, regions of interest (ROI) are indicated by abbreviations. F is frontal CSF, T is temporal CSF, P is parietal CSF, O is occipital CSF, aLV is anterior lateral ventricle, pLV is posterior lateral ventricle, and HippCSF is peri-hippocampal CSF.

Researchers validated the constructed learning model. Meanwhile, in the validation process, researchers used the cerebral cortex thickness and brain region volume predicted by the learning model of FIG. 4 as input data for the learning model of FIG. 5. Researchers performed 10-fold cross-validation. The validation results are shown in Table 5 below. In Table 5, 3D CIVET is the result of classification using volume values obtained from CIVET. Table 5 shows the results of classification using regions of interest segmented with the 2D image-based segmentation model and the results of classification using regions of interest segmented with the 3D image-based segmentation model. Validation compared the results predicted by the learning model with the ground truth. Performance indicators used AUC (area under the receiver operating characteristic curve) and AUPRC (area under the precision-recall curve).

TABLE 5

| | | | 2D image-based segmentation | | 3D image-based segmentation | |
|---|---|---|---|---|---|---|
| | | | model classification | | model classification | |
| | 3D CIVET | | performance | | performance | |
| Model Type | AUC | AUPRC | AUC | AUPRC | AUC | AUPRC |
| Model A1 | 0.791 | 0.772 | 0.783 | 0.767 | 0.784 | 0.769 |
| Model A2 | 0.767 | 0.737 | 0.738 | 0.730 | 0.772 | 0.735 |
| Model A3 | 0.779 | 0.744 | 0.746 | 0.737 | 0.779 | 0.738 |
| Model A4 | 0.780 | 0.746 | 0.746 | 0.738 | 0.773 | 0.734 |
| Model A5 | 0.805 | 0.772 | 0.801 | 0.776 | 0.800 | 0.776 |
| Model A6 | 0.800 | 0.784 | 0.766 | 0.766 | 0.777 | 0.763 |
| Model B1 | 0.924 | 0.903 | 0.894 | 0.893 | 0.900 | 0.892 |
| Model B2 | 0.882 | 0.870 | 0.838 | 0.837 | 0.884 | 0.881 |
| Model B3 | 0.910 | 0.897 | 0.872 | 0.866 | 0.902 | 0.899 |
| Model B4 | 0.930 | 0.913 | 0.908 | 0.899 | 0.916 | 0.907 |
| Model B5 | 0.863 | 0.870 | 0.854 | 0.865 | 0.862 | 0.874 |
| Model B6 | 0.912 | 0.882 | 0.885 | 0.879 | 0.885 | 0.872 |
| Model C1 | 0.945 | 0.903 | 0.920 | 0.897 | 0.934 | 0.899 |
| Model C2 | 0.926 | 0.899 | 0.877 | 0.869 | 0.937 | 0.927 |
| Model C3 | 0.935 | 0.915 | 0.893 | 0.886 | 0.937 | 0.929 |
| Model C4 | 0.944 | 0.913 | 0.919 | 0.905 | 0.942 | 0.917 |
| Model C5 | 0.903 | 0.910 | 0.905 | 0.914 | 0.904 | 0.915 |
| Model C6 | 0.935 | 0.893 | 0.907 | 0.889 | 0.921 | 0.887 |

Looking at Table 5, overall, the difference in performance between the results using 3D CIVET and the results using the segmentation model was not significant. In addition, it can be seen that the results of classification using the volume of regions of interest extracted using the 2D image-based segmentation model are sufficiently significant for calculating dementia-related information. Looking at Table 5, Model A, Model B, and Model C all showed significant performance, and overall, models using additional clinical information showed slightly higher performance. Furthermore, looking at Table 5, models using anterior lateral ventricle region (aLV), posterior lateral ventricle region (pLV), and peri-hippocampal cerebrospinal fluid region (hipp CSF) had relatively higher performance.

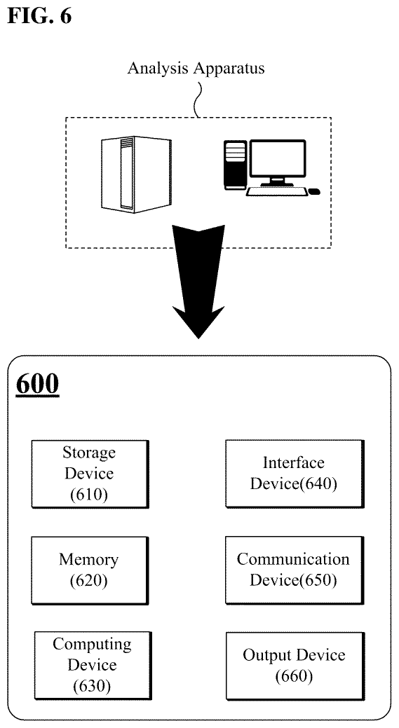

FIG. 6 is an example of an analysis apparatus (600) that derives dementia-related information of a subject using brain CT images. The analysis apparatus (600) may be physically implemented in various forms. For example, the analysis apparatus (600) may take the form of a computer device such as a PC, a smart device, a network server, or a data processing dedicated chipset. Meanwhile, the analysis apparatus (600) may be connected to brain imaging equipment or may be an integrated device.

The analysis apparatus (600) may include a storage device (610), memory (620), computing device (630), interface device (640), communication device (650), and output device (660).

The storage device (610) may store the subject's brain CT image generated by CT imaging equipment.

The storage device (610) may store the subject's clinical information. Clinical information may include at least one of age, gender, and APOE4 genotype.

The storage device (610) may store a segmentation model for extracting regions of interest from brain CT images (slices).

The storage device (610) may store a learning model that predicts the volume of the corresponding region of interest based on information about the region of interest. A learning model that predicts the volume of a specific brain region of interest is referred to as a first learning model. The region of interest includes at least one of frontal cerebrospinal fluid region, temporal cerebrospinal fluid region, parietal cerebrospinal fluid region, occipital cerebrospinal fluid region, anterior lateral ventricle region, posterior lateral ventricle region, and peri-hippocampal cerebrospinal fluid region. As described above, the first learning model may be prepared for each region of interest.

In addition, the storage device (610) may store a learning model that derives dementia-related information using the volume of regions of interest. A learning model that derives dementia-related information is referred to as a second learning model. As described above, the second learning model may be implemented as various models according to the type of input data. The second learning model may be at least one of various types such as (i) a model using only the volume of regions of interest as input data (Model A), (ii) a model using the volume of regions of interest and clinical information (age, gender) as input data (Model B), or (iii) a model using the volume of regions of interest and clinical information (age, gender, and APOE4) as input data (Model C). In addition, the region of interest used by the learning model may be all 7 regions of interest or some of the regions of interest as described in Table 3.

The dementia-related information inferred by the second learning model may be at least one of information such as dementia onset status, dementia risk, dementia probability, dementia-related score, dementia prognosis prediction, degree of brain atrophy, beta-amyloid (amyloid-β, Aβ) positivity, tau protein positivity, and brain age. The dementia-related information may vary depending on the type of training data (correct answer) used by the second learning model.

The memory (620) may store data and information generated in the process of the analysis apparatus (600) calculating dementia-related information from brain CT images.

The interface device (640) is a device that receives certain commands and data from the outside. The interface device (640) may receive the subject's brain CT image from a physically connected input device or external storage device. The interface device (640) may also transmit dementia-related information predicted based on brain CT images to external objects.

The interface device (640) may include a controller corresponding to each input/output device, a device driver that controls the operation of the controller, and a kernel I/O subsystem that integrally manages input/output control requests from the device driver. The kernel I/O subsystem stores input/output requests from the device driver in a queue and schedules those requests based on request priority or device status. For example, the input/output devices may include any of the following: keyboard, mouse, touchscreen, camera, Small Computer System Interface (SCSI) device, Peripheral Component Interconnect (PCI) bus-based device, or ATA Packet Interface (ATAPI) device.

The communication device (650) refers to a configuration that receives and transmits certain information through a wired or wireless network. The communication device (650) may receive the subject's brain CT image from external objects. The communication device (650) may also transmit dementia-related information predicted based on brain CT images to external objects such as user terminals.

The communication device (650) may include a transmitter and a receiver, and specifically may be configured with circuits and antennas for signal transmission control.

The interface device (640) may be a device that receives data received by the communication device (650) internally.

The output device (660) is a device that outputs certain information. The output device (660) may output interfaces necessary for the data processing process, brain images, regions of interest distinguished in brain images, dementia-related information derived based on regions of interest, etc.

The computing device (630) may uniformly preprocess the subject's brain CT image. For example, the computing device (630) may normalize the brain CT image to a certain size or resolution.

The computing device (630) may extract regions of interest by inputting the subject's brain CT image into a pre-trained segmentation model. The computing device (630) may extract regions of interest by inputting the subject's brain CT image into a pre-trained segmentation model in slice units.

The region of interest may be at least one of frontal cerebrospinal fluid region, temporal cerebrospinal fluid region, parietal cerebrospinal fluid region, occipital cerebrospinal fluid region, anterior lateral ventricle region, posterior lateral ventricle region, and peri-hippocampal cerebrospinal fluid region.

The computing device (630) may extract size information about the region(s) of interest. The computing device (630) may derive the total number of pixels for each region of interest. The computing device (630) may estimate the volume of the corresponding region of interest based on the number of pixels in the region of interest for each region of interest. The computing device (630) may derive the volume of the corresponding region of interest based on the number of pixels in the corresponding region of interest using the aforementioned first learning model. The computing device (630) may estimate the volume for each of multiple regions of interest using the first learning model constructed for each region of interest.

The computing device (630) may derive the subject's dementia-related information by inputting the volume of the region(s) of interest predicted by the first learning model into the second learning model. For example, the computing device (630) may derive the subject's dementia-related information by inputting the volume of at least one region among frontal cerebrospinal fluid region, temporal cerebrospinal fluid region, parietal cerebrospinal fluid region, occipital cerebrospinal fluid region, anterior lateral ventricle region, posterior lateral ventricle region, and peri-hippocampal cerebrospinal fluid region into the second learning model. In addition, the computing device (630) may derive the subject's dementia-related information by further inputting clinical information (at least one of age, gender, and APOE4 genotype) in addition to the volume of the region of interest into the second learning model. This process is as described in FIG. 5.

The computing device (630) may be a device such as a processor, AP, or chip with embedded programs that processes data and performs certain operations.

In addition, the brain CT image analysis method or dementia-related information calculation method as described above can be implemented as a program (or application) including executable algorithms that can be executed on a computer. The program may be provided by being stored in a temporary or non-transitory computer readable medium.

A non-transitory readable medium refers to a medium that stores data semi-permanently and can be read by a device, rather than a medium that stores data for a short moment such as registers, cache, memory, etc. Specifically, the various applications or programs described above may be stored and provided in non-transitory readable media such as CD, DVD, hard disk, Blu-ray disk, USB, memory card, ROM (read-only memory), PROM (programmable read only memory), EPROM (Erasable PROM, EPROM) or EEPROM (Electrically EPROM) or flash memory.

Temporary readable media refer to various RAMs such as Static RAM (SRAM), Dynamic RAM (DRAM), Synchronous DRAM (SDRAM), Double Data Rate SDRAM (DDR SDRAM), Enhanced SDRAM (ESDRAM), Synclink DRAM (SLDRAM), and Direct Rambus RAM (DRRAM).

This embodiment and the drawings attached to this specification merely clearly show part of the technical ideas included in the aforementioned technology, and it is obvious that all modified examples and specific embodiments that can be easily inferred by those skilled in the art within the scope of the technical ideas included in the specification and drawings of the aforementioned technology are included in the scope of rights of the aforementioned technology.

The non-transitory computer readable medium refers to a medium that stores data semi-permanently (e.g., the storage device 610) and is capable of being read by a device, rather than a medium that stores data for a short period of time, such as a register, cache, or memory. Specifically, the various applications or programs described above may be provided by being stored in the non-transitory computer readable medium such as a CD, a DVD, a hard disk, a Blu-ray disk, a USB, a memory card, a read-only memory (ROM), a programmable read only memory (PROM), an erasable PROM (EPROM), an electrically EPROM (EEPROM), or a flash memory.

The transitory computer readable medium refers to various types of RAM such as a static RAM (SRAM), a dynamic RAM (DRAM), a synchronous DRAM (SDRAM), a double data rate SDRAM (DDR SDRAM), an enhanced SDRAM (ESDRAM), a synclink DRAM (SLDRAM), and a direct Rambus RAM (DRRAM).

Various examples and aspects of the present disclosure are described below. These are provided as examples, and do not limit the scope of the present disclosure.

The description herein has been presented to enable any person skilled in the art to make, use and practice the technical features of the present disclosure, and has been provided in the context of one or more particular example applications and their example requirements. Various modifications, additions and substitutions to the described embodiments will be readily apparent to those skilled in the art, and the principles described herein may be applied to other embodiments and applications without departing from the scope of the present disclosure. The description herein and the accompanying drawings provide examples of the technical features of the present disclosure for illustrative purposes. In other words, the disclosed embodiments are intended to illustrate the scope of the technical features of the present disclosure. Thus, the scope of the present disclosure is not limited to the embodiments shown, but is to be accorded the widest scope consistent with the claims. The scope of protection of the present disclosure should be construed based on the following claims, and all technical features within the scope of equivalents thereof should be construed as being included within the scope of the present disclosure.

What is claimed is:

1. A method of deriving dementia-related information using volume predicted from brain CT, comprising:
receiving, by an analysis apparatus, a brain CT (Computed Tomography) image of a subject;
extracting, by the analysis apparatus, regions of interest by inputting the brain CT image into a pre-trained segmentation model;
predicting, by the analysis apparatus, a volume of at least one region among the regions of interest by inputting pixel information of the at least one region into a pre-trained first learning model; and
calculating, by the analysis apparatus, dementia-related information of the subject by inputting the volume of the at least one region into a pre-trained second learning model,
wherein the regions of interest include a plurality of regions selected from frontal cerebrospinal fluid region, temporal cerebrospinal fluid region, parietal cerebrospinal fluid region, occipital cerebrospinal fluid region, anterior lateral ventricle region, posterior lateral ventricle region, and peri-hippocampal cerebrospinal fluid region.

2. The method of claim 1, wherein the first learning model comprises a plurality of learning models prepared in advance for each of the regions of interest.

3. The method of claim 1, wherein the second learning model further receives at least one information of age of the subject, gender of the subject, and APOE4 genotype of the subject to derive dementia-related information of the subject.

4. The method of claim 1, wherein the dementia-related information is one of dementia onset status, dementia risk, dementia probability, dementia-related score, dementia prognosis prediction, degree of brain atrophy, beta-amyloid (amyloid-β, Aβ) positivity, tau protein positivity, and brain age.

5. The method of claim 1, wherein the segmentation model comprises a plurality of models prepared in advance for each of the regions of interest.

6. An analysis apparatus for deriving dementia-related information using volume predicted from brain CT, comprising:
an interface device for receiving a brain CT (Computed Tomography) image of a subject;
a storage device for storing a segmentation model for extracting regions of interest from a brain CT image, a first learning model for receiving brain region of interest information and predicting volume, and a second learning model for calculating dementia-related information; and
a computing device for extracting regions of interest by inputting the received brain CT image into the segmentation model, predicting a volume of at least one region among the regions of interest by inputting pixel information of the at least one region into the first learning model, and calculating dementia-related information of the subject by inputting the volume of the at least one region into the second learning model,
wherein the regions of interest include a plurality of regions selected from frontal cerebrospinal fluid region, temporal cerebrospinal fluid region, parietal cerebrospinal fluid region, occipital cerebrospinal fluid region, anterior lateral ventricle region, posterior lateral ventricle region, and peri-hippocampal cerebrospinal fluid region.

7. The analysis apparatus of claim 6, wherein the segmentation model comprises a plurality of models prepared in advance for each of the regions of interest.

8. The analysis apparatus of claim 6, wherein the first learning model comprises a plurality of learning models prepared in advance for each of the regions of interest.

9. The analysis apparatus of claim 6, wherein the second learning model further receives at least one information among age of the subject, gender of the subject, and APOE4 genotype of the subject to derive dementia-related information of the subject.

10. The analysis apparatus of claim 6, wherein the dementia-related information is one of dementia onset status, dementia risk, dementia probability, dementia-related score, dementia prognosis prediction, degree of brain atrophy, beta-amyloid (amyloid-β, Aβ) positivity, tau protein positivity, and brain age.
